(19) United States
(12) Patent Application Publication
(10) Pub. No.: US 2010/0075345 A1
(43) Pub. Date: Mar. 25, 2010

(54) METHOD OF SCREENING

(76) Inventor: Benjamin Thomas Kile, Carlton (AU)

Correspondence Address: KLAUBER & JACKSON, 411 HACKENSACK AVENUE, HACKENSACK, NJ, 07601, US

(21) Appl. No.: 12/560,403

(22) Filed: Sep. 15, 2009

Related U.S. Application Data
(63) Continuation of application No. PCT/AU2008/000403, filed on Mar. 20, 2008.

(30) Foreign Application Priority Data
Mar. 20, 2007 (AU) 2007901452
Aug. 10, 2007 (AU) 2007904304

(51) Int. Cl. G01N 33/53

(52) U.S. Cl. 435/7.23; 435/7.21; 435/7.24

(57) ABSTRACT

A method of screening for a molecule which decreases apoptosis of a cell, comprising: i) combining the candidate molecule and an assay cell; and ii) determining the change in survival of the assay cell in the presence of the molecule relative to a control. In one embodiment, the assay cell is treated with an apoptosis inducing agent prior to or between steps i) and ii). In one example, the treating agent reduces the level or activity of a pro-survival member of the Bcl-2 protein family, such as Bcl-x_{L} or Mcl-1. In another embodiment, the level or activity of at least one pro-survival member of the Bcl-2 family is reduced in the cell of step i). In some embodiments, this is independent of any effect of the candidate molecule or apoptosis promoting agent. By reducing the level or activity of one or more pro-survival Bcl-2 protein family members in the assay cell, the cell will undergo apoptosis mediated inter alia by Bak or Bax or Bak and Bax unless it is rescued by the candidate molecule.

### RELATED APPLICATIONS

The present application is a Continuation of co-pending PCT Application No. PCT/AU2008/000403 filed Mar. 20, 2008, which in turn, claims priority from Australian Application No. 2007901452 filed Mar. 20, 2007 and Australian Application No. 2007904304 filed Aug. 10, 2007. Applicants claim the benefits of 35 U.S.C. §120 as to the PCT application and priority under 35 U.S.C. §119 as to the said Australian applications, and the entire disclosures of all applications are incorporated herein by reference in their entireties.

### FIELD

The present invention relates to apoptosis and the Bcl-2 family of proteins. In particular, the present invention relates to a method of screening for molecules which modulate cellular survival and/or apoptosis and/or the level and/or activity of a member of the Bcl-2 family of proteins.

### BACKGROUND

Bibliographic details of the publications referred to by author in this specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The survival of multicellular organisms depends on the correct and co-ordinated functioning of various cell types. During the initial stages of development, viability of the organism depends on the selection and differentiation of cells in the various tissues. At later stages, the maintenance of the organism requires a specific cellular adaptability. As examples, blood cells are constantly renewed from hematologic precursors. Lymphocytes or reproductive cells show rapid expansion in response to immediate requirements. On the other hand, neural cells display a limited capability for renewal, and many neurons survive and persist throughout the life of the individual. For each cell type, control of the number of cells is the result of a balance between cell proliferation and cell death.

One process of cell death is apoptosis. Apoptosis, or programmed cell death, is an organism's normal method of disposing of damaged, unwanted, or unneeded cells. However sometimes, such as when the organism has a disease, the rate of apoptosis of one or more cell types of the body is affected. For example, an aberrant regulation of apoptosis, leading to too much or too little cell suicide, probably contributes to such varied disorders in humans as cancer, AIDS, Alzheimer's disease and rheumatoid arthritis.

The molecular regulation of apoptosis has been characterised in detail over the last 20 years and has been found to be executed by a family of aspartate-specific cysteine proteases (caspases). One major pathway which regulates apoptosis is the Bcl-2 protein family pathway, which plays a central role in regulating developmentally programmed and stress induced cell deaths. One subclass of this family, including the proteins Bcl-2, Bcl-x_{L}, Bcl-w, Mcl-1, A1 and Bcl-B, promotes cellular survival. These proteins maintain the survival of a cell until their activity is reduced or neutralised by direct binding of the pro-apoptotic family members, such as the proteins Bim, Bad, or Bid. The precise biochemical action of the pro-survival proteins is controversial although it is likely that they control the action of a second class of pro-apoptotic family members, the multi-domain proteins Bax and Bak. These proteins play an essential role in mediating apoptosis, probably by damaging intracellular membranes such as the outer mitochondrial membrane, thereby precipitating the release of pro-apoptogenic factors such as cytochrome c, normally sequestered within the organelles, into the cytoplasm to promote caspase activation.

As excessive or impaired apoptosis characterise many diseases or unwanted conditions, a need exists for molecules which modulate apoptosis and/or a member of the Bcl-2 family of proteins.

### SUMMARY OF THE BROAD EMBODIMENTS

Each embodiment in this specification is to be applied mutatis mutandis to every other embodiment unless expressly stated otherwise.

Accordingly, in one aspect the invention provides a method of screening for a molecule which modulates apoptosis of a cell, comprising:

i) combining the molecule and the cell; and

ii) identifying modulation of a Bcl-2 family protein of the cell,

wherein modulation of the Bcl-2 family protein indicates that the molecule modulates apoptosis of the cell. In a preferred embodiment, the molecule decreases apoptosis.

In another aspect the invention provides a method of screening for a molecule which modulates a Bcl-2 family protein of a cell, comprising:

i) combining the molecule and the cell; and

ii) identifying whether apoptosis of the cell is modulated,

wherein modulation of apoptosis indicates that the molecule modulates the Bcl-2 family protein. In a preferred embodiment, the molecule decreases apoptosis.

In yet another aspect, the invention provides a method of screening for a molecule which decreases apoptosis of a cell, comprising:

i) combining a candidate molecule with an assay cell; and

ii) determining the change in survival of the assay cell in the presence of the molecule relative to a control.

In some embodiments the methods of the invention further comprise the step, prior to or between steps i) and ii), of treating the cell to induce apoptosis. The cell may be treated with an agent which reduces the level and/or activity of a pro-survival member of the Bcl-2 protein family, such as Bcl-x_{L} and/or Mcl-1. In some embodiments the cell is treated with an agent which reduces or enhances the level and/or activity of a pro-apoptotic member of the Bcl-2 family, such as Bak and/or Bax.

In some embodiments the level and/or activity of at least one pro-survival member of the Bcl-2 family is reduced in the cell of step i). The level and/or activity of between one and six members of the Bcl-2 family selected from the group consisting of Bcl-x_{L}, Bcl-2, Bcl-w, Mcl-1, A1 and Bcl-B may be reduced in the cell of step i). The level and/or activity of Bcl-x_{L} and/or Mcl-1 may be reduced. In some embodiments the level and/or activity of at least one pro-apoptosis member of the Bcl-2 protein family is reduced in the cell of step i), for example the level and/or activity of Bak and/or Bax may be reduced. In some embodiments, the apoptosis inducing agent targets the gene, RNA or protein of the Bcl-2 family member using methods known in the art.

In some embodiments the level and/or activity of Mcl-1 and Bak are reduced. In other embodiments the level and/or activity of Mcl-1 and Bax are reduced. In some embodiments the level and/or activity of Mcl-1 and Bak are enhanced. In other embodiments the level and/or activity of Mcl-1 and Bax are enhanced. Thus, the cells may be genetically modified or treated with agents whereby the level and/or activity of at least one pro-survival or pro-apoptotic molecule is modified independently of an effect of the candidate molecule or apoptosis inducing agent. In some embodiments, this step enhances the sensitivity of the assay cell for identifying a cell survival agent.

In some embodiments the molecule is an agonist. In other embodiments the molecule is an antagonist. In some embodiments, the molecule is an antagonist of Bak or Bax or Bak and Bax.

In some embodiments apoptosis is increased. In other embodiments apoptosis is decreased.

In some embodiments the method occurs in vitro. In other embodiments the method occurs in vivo.

In some embodiments the cell is a fibroblast, myeloid or lymphoid cell.

In some embodiments, the method comprises screening for a molecule which enhances the survival, lifespan or viability of mammalian cells. In an illustrative embodiment the method comprises: (i) combining the molecule with a cell; (ii) contacting the cell with one or more agents that antagonise pro-survival Bcl-2 family molecules in the cell and induce/s apoptosis; (iii) determining the change in survival (viability, lifespan, half-life) of cells in the presence of the molecule relative to a control; and (iv) selecting a molecule which enhances cell survival (viability, half-life). In some embodiments, the method further comprises combining the selected molecule from (iv) with a target mammalian cell to determine the change in cell survival (viability, half-life) of the cell in the presence of the molecule relative to controls.

To facilitate screening, in some embodiments, the cell is modified to enhance its sensitivity to an apoptosis inducing agent, such as by reducing the level or activity of one or more pro-survival Bcl-2 family members. In other embodiments, the cell is modified to lack one or more pro-survival Bcl-2 family members by gene disruption. In an illustrative embodiment, the cell is an Mcl-1 deficient cell from a multicellular organism and the agent is a Bcl-x_{L} antagonist. In still other embodiments, the method comprises identifying modulation of a Bcl-2 family protein in the cell.

In another embodiment, cellular assays are used to identify compounds that maintain platelet viability.

The subject methods comprise incubating cells that are sensitive to apoptosis inducing agents in the presence of a compound to be tested, then contacting the cells with an apoptosis inducing agent and determining the presence of live cells that have not undergone apoptosis. In some embodiments, the cells are sensitive to antagonists of one or more members of the Bcl-2 family (including, for example, Bcl-2, Bcl-x_{L}, Bcl-w, Mcl-1 and A1) such as BH3 domain mimicking agents. In other embodiments, the cells are sensitive to Bcl-x_{L} or Mcl-1 antagonists. In another embodiment the Bcl-x_{L} antagonist is ABT-737.

In some embodiments, the cells are a fibroblast, neural cell, epithelial cell, endothelial cell, stem cell, hepatocyte, myoblast, osteoblast, osteoclast, lymphocyte, keratinocyte, mesothelial cell, germ cell, muscle cell or fibroblasts such as mouse embryo fibroblasts (MEFs). In some embodiments, the mammalian cell is a myeloid cell, lymphoid cell, neural cell, epithelial cell, endothelial cell, stem or progenitor cell, hepatocyte, myoblast, osteoblast, osteoclast, lymphocyte, keratinocyte, melanocyte, mesothelial cell, germ cell, muscle cell, fibroblast, a transformed cell, a cancer cell.

In a preferred embodiment, the cells are cells in which the level or activity of Mcl-1 or Bel-x_{L} is down regulated either in part or in full, generated by methods known in the art. In some embodiments, Mcl-1 or Bel-x_{L} levels are down regulated using chemical, genetic or gene silencing (RNAi) methods. For example, Mcl-1 levels can be reduced using CDK inhibitors (e.g. R-roscovitine) or protein synthesis inhibitors (e.g. cyclohexamide). Genetic strategies include creation of loss of function alleles through deletion of all or part of a gene or through insertion of foreign DNA into a gene or through expression of a transgene from an exogeneous promoter. Conditional mutant technology may also be employed. Gene silencing offers a convenient procedure for inhibiting the function of genes. Mcl-1 antisense oligonucleotides are described, for example, in International Publication No. WO 2006/099667 incorporated herein in its entirety. Bel-x_{L} level or activity is conveniently reduced using ABT-737 or an equivalent BH3 domain mimicking agent.

Thus, in some embodiments, the invention provides a method of identifying compounds that maintain cellular viability comprising incubating cells that are sensitive to Bel-x_{L} or Mcl-1 antagonists in the presence of a compound to be tested, contacting said cells with a Bel-x_{L} or Mcl-1 antagonist and determining the presence of live cells indicating that the compound is capable of blocking Bel-x_{L} or Mcl-1 antagonist-inducing cell death and maintaining cell viability. One embodiment of the invention is described in Example 2. In another embodiment, the Bel-x_{L} antagonist is ABT-737 or an analogue thereof. In some embodiments, cells that are sensitive to Bel-x_{L} antagonists are Mcl-1 deficient. In other embodiments, cells that are sensitive to Mcl-1 antagonists are Bel-x_{L} deficient. Compounds identified through initial screens are then tested to determine upon which targets they act. For example, compounds are tested in Mcl-1 null, Bax-null cells and Mcl-1 null, Bak-null cells to confirm that the compounds act via Bax or Bak, or other proapoptotic molecules or a further downstream target. If required, further downstream targets are then tested in this manner.

Accordingly, in some embodiments, the subject methods comprise combining the molecule with a cell deficient in one or more Bcl-2 family members selected from the group consisting of Bel-x_{L}, Bcl-2, Bel-w, Mcl-1, A1 (Bfl-1), Bel-B, Bak, Bax, Bok (Mtd), Bad, Bid, Bik (Blk), Hrk (DP5), BNIP3, Bim, Puma, Noxa, Mule (Lasu/ARF-BPI), and Bmf.

In some other embodiments, the methods further comprise combining the molecule with a cell and determining the change in survival of the cell in the presence of the molecule relative to a control. In a preferred embodiment, the cell is a mammalian cell. In an illustrative embodiment, the cell is selected from the group consisting of a myeloid cell, lymphoid cell, neural cell, epithelial cell, endothelial cell, stem or progenitor cell, hepatocyte, myoblast, osteoblast, osteoclast, lymphocyte, keratinocyte, melanocyte, mesothelial cell, germ cell, muscle cell, fibroblast, a transformed cell and a cancer cell. In another embodiment, the mammalian cell is a cell subject to enhanced apoptosis in an apoptosis mediated disease or condition.

In an illustrative but non-limiting embodiment, the anti-apoptotic agent is an agent identified in the herein disclosed cellular screen. The present invention provides for the use of the anti-apoptotic molecules identified herein in the manufacture of a medicament for the treatment of diseases and conditions characterised by debilitating or unwanted cellular apoptosis including those denoted herein. In an illustrative but non-limiting embodiment, the agent is selected from one of the corticosteroid molecules set out in FIG. 4 or comprises the general structure set out in FIG. 4. As described herein, in Example 3 and FIG. 4 these agents strongly inhibited killing in mammalian cells exposed to an apoptosis inducing amount of a Bcl-x_{L} antagonist.

### BRIEF DESCRIPTION OF THE FIGURES

FIG. 1 shows a diagram of the control of cell survival by the Bel-2 protein family. Bax and Bak are the essential mediators of apoptosis. In healthy cells, they are restrained by pro-survival Bel-2 proteins, namely Bel-2, Bcl-x_{L}, Bcl-w, Mcl-1, and A1. Damage signals inactivate the pro-survival proteins thereby unleashing Bax and Bak to cause cell death.

FIG. 2 shows a diagram of apoptosis signalling in fibroblasts. (A) In wild-type mouse embryo fibroblasts (MEFs), pro-apoptotic Bak is normally constrained by Bcl-x_{L} and Mcl-1. Inactivating Bcl-x_{L} with the BH3 mimetic compound ABT-737 does not cause cell death unless Mcl-1 is also inactivated. (B) In the constitutive absence of Mcl-1 MEFs will be highly sensitive to ABT-737.

FIG. 3 shows the screening strategy. In Mcl-1-deficient cells, ABT-737 kills potently. By pre-incubating such cells with diverse library molecules, a screen is conducted for molecules that can inhibit ABT-737-induced killing. Such molecules may act to block the action of ABT-737 or to directly block the action of the cell death mediators, Bax and Bak.

FIG. 4 is a structural representation of agents identified in the subject cellular screens for agents that enhance cellular viability, survival or life span.

### DETAILED DESCRIPTION OF THE PREFERRED EMBODIMENTS

Before describing the invention in detail it is to be understood that it is not limited to particularly exemplified methods, formulations, or components and may, of course, vary. It is also to be understood that the terminology used herein is for the purpose of describing particular embodiments of the invention only, is not intended to be limiting, and will be limited only by the appended claims.

All publications, patents and patent applications cited herein, whether supra or infra, are hereby incorporated by reference in their entirety. However, publications mentioned herein are cited for the purpose of describing and disclosing the protocols and reagents which are reported in the publications and which might be used in connection with the invention. Nothing herein is to be construed as an admission that the invention is not entitled to antedate such disclosure by virtue of prior invention.

Furthermore, the practice of the present invention employs, unless otherwise indicated, conventional molecular biology, cell biology, and cell culture, techniques within the skill of the art. Such techniques are well known to the skilled worker, and are explained fully in the literature. See, e.g., Sambrook et al. (eds), Molecular Cloning: A Laboratory Manual”, 2nd Ed., Cold Spring Harbor Laboratory Press; 1989; Hames et al. (eds), Nucleic Acid Hybridization, A Practical Approach, IRL Press, 1985; Gait (ed), Oligonucleotide Synthesis, Oxford IRL Press, 1984; Remington's Pharmaceutical Sciences, 17^{th} Edition, Mack Publishing Company, Easton, Pa., USA; Alberts et al., Molecular Biology of the Cell, 4th ed., New York and London: Garland Science, c2002; Lodish et al., Molecular Cell Biology”, 4th ed., New York: W. H. Freeman & Co., c2000.

It must be noted that, as used in the subject specification, the singular forms “a”, “an” and “the” include plural aspects unless the context clearly dictates otherwise. Thus, for example, reference to a “molecule” includes a single molecule, as well as two or more molecules; reference to “a cell” includes a single cell, as well as two or more cells; “a protein” includes a single protein or two or more proteins, and so forth.

Throughout the specification the word “comprise” and variations of the word, such as “comprising” and “comprises”, means “including but not limited to” and is not intended to exclude other additives, components, integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

The present invention is based on the discovery that screening for molecules which modulate apoptosis of a cell can identify molecules which modulate the level and/or activity of a member of the Bcl-2 family of proteins. Conversely, screening for molecules which modulate the level and/or activity of a member of the Bcl-2 family of proteins can identify molecules which modulate apoptosis.

In some embodiments the invention relates to the Bcl-2 family of proteins. Bcl-2 is the prototype for a family of mammalian genes and the proteins which they produce. The Bcl-2 family govern mitochondrial outer membrane permeabilisation (MOMP) and can either be pro-apoptotic (Bax, Bak, and Bok, among others) or pro-survival (including Bcl-2, Bcl-x_{L}, and Bcl-w). There are over 20 proteins in the Bcl-2 family and these are divided into three groups. Generally, group 1 members are pro-survival whereas groups 2 and 3 are pro-apoptotic. The members of the Bcl-2 family share one or more of the four characteristic domains of homology entitled the Bcl-2 homology (BH) domains, named BH1, BH2, BH3, and BH4. The Bcl-2 family has a general structure that consists of a central hydrophobic helix surrounded by amphipathic helices. Many members of the family have transmembrane domains. The site of action for the Bcl-2 family is mostly on the outer mitochondrial membrane. Within the mitochondria are apoptogenic factors (cytochrome c, Smac/DIABLO, Omi) that if released activate the caspases. Depending on their function, once activated, Bcl-2 proteins either promote the release of these factors, or keep them sequestered in the mitochondria. The exact mechanisms surrounding Bcl-2 regulated MOMP have yet to be elucidated, but it is believed that the multidomain, pro-apoptotic Bcl-2 proteins can activate MOMP directly, a process that is inhibited by the binding of anti-apoptotic Bcl-2 proteins. In contrast, the BH3-only pro-apoptotic Bcl-2 proteins activate MOMP indirectly by binding the anti-apoptotic Bcl-2 proteins, freeing the multidomain, pro-apoptotic Bcl-2 proteins to activate MOMP.

Pro-apoptotic members of the Bcl-2 family include Bak, Bok (Mtd), Bax, Bad, Bid, Bik (Blk), Hrk (DP5), BNIP3, Bim, Puma, Noxa, Mule (Lasu/ARF-BPI), and Bmf. Pro-survival members include Bcl-2, Bcl-x_{L}, Mcl-1, Bcl-w, Bcl-B, and A1 (Bfl-1 in humans). Bcl-2 can occur as Bcl-2 alpha or Bcl-2 beta, two alternatively spliced forms which solely differ in their carboxyl termini.

In some embodiments the invention relates to apoptosis. “Apoptosis”, also called programmed cell death, it is a signalling pathway that leads to cellular suicide in an organized manner. Several factors and receptors are specific to the apoptotic pathway. The net result is that cells shrink, develop blebs on their surface, and their nucleic acids undergo fragmentation. In multicellular organisms, apoptosis is mediated by caspases, which trigger cell death by cleaving specific proteins in the cytoplasm and nucleus. Caspases exist in all cells as inactive precursors, or procaspases, which are usually activated by cleavage by other caspases, producing a proteolytic caspase cascade.

In some embodiments the apoptosis is Bak and/or Bax mediated.

The molecule screened by a method of the invention may be any molecule comprising two or more atoms held together by a chemical bond. The molecule may be a drug, small or large chemical molecule, a protein (such as an antibody) or derivative thereof, a peptide including a modified peptide such as a constrained peptide or foldamer, a lipid, a carbohydrate, or a nucleic acid molecule including an antisense or other gene silencing molecule, or a mimetic thereof. The molecule may be a naturally occurring or non-naturally occurring molecule and may be located in a naturally-produced library, chemically-produced library, combinatorial library, phage display library, or in vitro translation-based library.

Where the molecule is a “drug”, this refers to a chemical compound that induces a desired pharmacological effect and includes the active agent per se as well as pharmaceutically acceptable and pharmaceutically active salts, esters, amides, prodrugs, enantiomers, metabolites, and analogues of the active agent.

Where the molecule is a “small or large molecule”, this refers to a small or large natural or synthetically derived organic or inorganic molecule. A “small molecule” has a molecular weight below about 500 Daltons. A large molecule has a molecular weight above 500 Daltons.

Where the molecule is a “protein”, this refers to a polymer of amino acids linked via peptide bonds which may be composed of two or more polypeptide chains. The term “derivative” includes proteins and peptides with one or several amino acid residues substituted by naturally-occurring or synthetic amino acid homologues of the 20 standard amino acids. Synthetic amino acid homologues include both D- and L-forms of any other amino acid residues whether found in a protein, found in nature or synthetically produced. Examples of such homologues are 4-hydroxyproline, 5-hydroxylysine, 3-methylhistidine, homoserine, ornithine, β-alanine and 4 aminobutanoic acid, beta-alanine, ornithine, norleucine, norvaline, hydroxyproline, thyroxine, gamma-amino butyric acid, homoserine, citrulline, and the like.

As used herein, natural amino acid residues are the 20 amino acid residues commonly found in proteins (i.e. alanine, aspartic acid, asparagine, arginine, cysteine, glycine, glutamine, glutamic acid, histidine, isoleucine, leucine, lysine, methionine, phenylalanine, proline, serine, threonine, tyrosine, tryptophan and valine), and include both the D- and L-forms of such amino acids.

The protein may be an antibody since it is known in the art that an antibody which specifically binds a protein or peptide can act as an antagonist or agonist of the protein or peptide. Thus, an antibody which specifically binds a member of the Bcl-2 family of proteins may modulate apoptosis. The term “antibody” is used in the broadest sense and specifically covers, without limitation, intact monoclonal antibodies, polyclonal antibodies, multispecific antibodies (e.g., bispecific antibodies) formed from at least two intact antibodies, and antibody fragments, so long as they specifically bind a member of the Bcl-2 family of proteins. Some antibodies have the capacity for intracellular transmission, such as cartilage fish-derived antibodies. These are described for example in International Patent Publication No. WO 2005/118629.

An antibody that “specifically binds” or is “specific for” a particular protein or peptide or an epitope on a particular protein or peptide is one that binds to that particular protein, peptide, or epitope on a particular protein or peptide without substantially binding to any other protein peptide, or epitope.

Where the molecule is a “lipid”, this refers to any of a group of fats and fatlike compounds, including sterols, fatty acids, and many other substances, waxes, phosphatides, cerebrosides, and related and derived compounds.

Where the molecule is a “carbohydrate”, this refers to organic compounds made up of a chain or ring of carbon atoms to which hydrogen and oxygen atoms are attached in a defined ratio (2:1). Carbohydrates vary from simple sugars containing from three to seven carbon atoms to very complex polymers.

Where the molecule is a “nucleic acid molecule”, this refers to a double or single stranded nucleic acid molecule and includes nucleic acid molecules such as sense and antisense DNA (gDNA, cDNA), RNA (sense RNA, antisense RNA, mRNA, tRNA, rRNA, small interfering RNAs (siRNAs), micro RNAs (miRNAs), small nucleolar RNAs (snRNAs), ribozymes, aptamers, DNAzymes, or other ribonuclease-type complexes.

Antisense nucleic acid molecules have been shown to be potent and specific inhibitors of the function of a gene or its associated gene products. Thus an antisense molecule may modulate the level and/or activity of a member of the Bcl-2 family of proteins in a cell. Antisense molecules have a sequence complementary to the sequence of another nucleic acid molecule, such as a nucleic acid molecule encoding a member of the Bcl-2 protein family.

Aptamers are also contemplated. DNA and RNA aptamers can substitute for monoclonal antibodies in various applications. They are nucleic acid molecules having specific binding affinity to non-nucleic acid or nucleic acid molecules through interactions other than classic Watson-Crick base pairing. Aptamers are described, for example, in U.S. Pat. Nos. 5,475,096, 5,270,163, 5,589,332, 5,589,332, and 5,741,679.

Where the molecule is a “mimetic”, this includes carbohydrate, nucleic acid, or protein or peptide mimetics and is intended to refer to a substance which has conformational features allowing the substance to perform as a functional analogue. A peptide mimetic may be a peptide containing molecule that mimic elements of protein secondary structure (Johnson et al., Peptide Turn Mimetics in Biotechnology and Pharmacy, Pezzuto et al., eds Chapman and Hall, New York, 1993). Peptide mimetics may be identified by screening random peptides libraries such as phage display or combinatorial libraries for peptide molecules which mimic the functional activity of Bcl-2 polypeptides. Alternatively, mimetic design, synthesis and testing may be employed. The recognition of carbohydrates and lipids by proteins is an important event in many biological systems and the development of chemotherapeutics based on carbohydrate and/or lipid-mimics which can disrupt specific recognition processes is a rapidly emerging field. Nucleic acid mimetics include, for example, RNA analogues containing N3′-P5′ phosphoramidate internucleotide linkages which replace the naturally occurring RNA O3′-P5′ phosphodiester groups. Enzyme mimetics include catalytic antibodies or their encoding sequences, which may also be humanised.

The molecule identified by a method of the invention may modulate apoptosis. As used herein the term “modulate” means changed or adjusted. Thus the rate of apoptosis of the cell may be changed or adjusted. The rate of apoptosis may be increased or decreased. That is, the life of the cell may be made greater or lesser. Alternatively or in addition, the level and/or activity of a member of the Bcl-2 family of proteins may be modulated and may be increased or decreased. That is, the level and/or activity of the Bcl-2 family member may be made greater or lesser.

The molecule may modulate apoptosis and/or the level and/or activity of a member of the Bcl-2 family directly or indirectly. For example, the molecule may bind to a member of the Bcl-2 protein family. Alternatively the molecule may bind to another molecule which in turn binds to a member of the Bcl-2 protein family. For example, the molecule may indirectly modulate apoptosis and/or the level and/or activity of a member of the Bcl-2 protein family by binding to ABT-737. The small molecule ABT-737 is a BH3 mimetic drug that antagonizes pro-survival Bcl-x_{L}. It selectively targets Bcl-2, Bcl-x_{L} and Bcl-w but not the other pro-survival proteins Mcl-1 or A1. Alternatively, the molecule may modulate apoptosis by binding to another molecule downstream from the Bcl-2 protein family, such as a caspase.

The molecule may be an agonist or antagonist of a member of the Bcl-2 protein family. As used herein the term “agonist” refers to a molecule that improves the activity of a different molecule. The term “antagonist” refers to a molecule that counteracts the action of another. Thus the molecule may upregulate or downregulate apoptosis and/or the level and/or activity of a member of the Bcl-2 family of proteins.

The molecule identified by a method of the invention may have use generally in preserving or maintaining cell viability, and especially mammalian cell viability, for example, in the treatment or prevention of an apoptosis mediated disease or unwanted condition including, cytopenia, an inflammatory disease, an autoimmune disease, a destructive bone disorder, a proliferative disorder, an infectious disease, a degenerative disease, a disease associated with cell death, an excess dietary alcohol intake disease, a viral mediated disease, uveitis, inflammatory peritonitis, osteoarthritis, pancreatitis, asthma, adult respiratory distress syndrome, glomerulonephritis, rheumatoid arthritis, systemic lupus erythematosus, scleroderma, chronic thyroiditis, Grave's disease, autoimmune gastritis, diabetes, autoimmune hemolytic anemia, autoimmune neutropenia, thrombocytopenia, chronic active hepatitis, myasthenia gravis, inflammatory bowel disease, Crohn's disease, psoriasis, atopic dermatitis, scarring, graft vs host disease, organ transplant rejection, osteoporosis, leukemias and related disorders, myelodysplastic syndrome, multiple myeloma-related bone disorder, acute myelogenous leukemia, chronic myelogenous leukemia, metastatic melanoma, Kaposi's sarcoma, multiple myeloma, haemorrhagic shock, sepsis, septic shock, burns, Shigellosis, Alzheimer's disease, Parkinson's disease, Huntington's disease, prion disease, cerebral ischemia, epilepsy, myocardial, ischemia, acute and chronic heart disease, myocardial infarction, congestive heart failure, atherosclerosis, coronary artery bypass graft, spinal muscular atrophy, amyotrophic lateral sclerosis, multiple sclerosis, HIV-related encephalitis, aging, alopecia, neurological damage due to stroke, ulcerative colitis, traumatic brain injury, spinal cord injury, hepatitis-A, hepatitis-B, hepatitis-C, hepatitis-D, hepatitis-E, hepatitis-G, other forms of viral hepatitis, drug (e.g. paracetamol)-induced liver disease, yellow fever, dengue fever, Japanese encephalitis, liver disease, alcoholic hepatitis, renal disease, polycystic kidney disease, H. pylori-associated gastric and duodenal ulcer disease, HIV infection, tuberculosis, meningitis, and to treat complications associated with coronary artery bypass grafts. One embodiment of the present invention contemplates methods wherein the cell tested is a mammalian cell subject to enhanced apoptosis in an apotosis mediated condition such as those denoted supra.

More specifically, a molecule identified by a method of the invention may be used to preserve organ viability, for example, in kidneys, heart valves, lungs, liver, skin, corneas, veins and other vessels, bones, tendons, and musculo skeletal tissue, pancrease, intestines etc. In some embodiments, a molecule so identified is used to prolong platelet survival in patients or in blood bank storage, as well as to treat or prevent myocardial infarcts, reperfusion injuries, thrombotic strokes to minimize loss of neuronal tissues, prevent gut toxicity (mucositis) following high-dose chemotherapy and total body radiation, hepatitis and other forms of liver failures, inflammatory diseases that lead to tissue loss e.g. rheumatoid arthritis, anemias, neutropenias, infertility due to loss of sperm viability, and premature greying due to loss of melanocytes (cells for hair pigmentation). One embodiment of the present invention contemplates methods wherein the cell tested is a mammalian cell subject to enhanced apoptosis in an apotosis mediated condition such as those denoted supra.

The cell used to identify modulation of the level and/or activity of a member of the Bcl-2 family of proteins and/or apoptosis and the cell to be treated or whose life span is to be maintained or enhanced may be any cell which comprises one or more members of the Bcl-2 protein family, that is, any cell of a multicellular organism. In a preferred embodiment, the cell is a mammalian cell. The cell may be from any multicellular organism as members of the Bcl-2 family of proteins, or homologues thereof, are found in organisms such as C. elegans, mice, and humans. Thus the cell may be from a human or a mammal of economical importance and/or social importance to humans, for instance, carnivores other than humans (such as cats and dogs), swine (pigs, hogs, and wild boars), ruminants (such as cattle, oxen, sheep, giraffes, deer, goats, bison, and camels), horses, and birds including those kinds of birds that are endangered, kept in zoos, and fowl, and more particularly domesticated fowl, e.g., poultry, such as turkeys, chickens, ducks, geese, guinea fowl, and the like, as they are also of economical importance to humans. The term does not denote a particular age. Thus, cells from both adult and newborn organisms are intended to be covered.

The cell may be any cell having a nucleus including, without limitation, a fibroblast, neural cell, epithelial cell, endothelial cell, stem cell, hepatocyte, myoblast, osteoblast, osteoclast, lymphocyte, keratinocyte, mesothelial cell, and muscle cell. Alternatively the cell may be anuclear, that is, without a nucleus, and thus have no DNA. An example of an anuclear cell is a platelet (thrombocyte). In some embodiments, the cell is not a platelet cell.

In some embodiments the cell is deficient in one or more pro-survival members of the Bcl-2 protein family. In other embodiments the cell is deficient in one or more pro-apoptotic members of the Bcl-2 protein family. In some embodiments the cells are Mcl-1 deficient cells.

The “assay” cell used in some embodiments of the methods disclosed herein may be the same cell or a different cell to the cell which is treated therapeutically or prophylactically with the molecules identified with the subject methods. A cell includes reference to multiple cells such as are found in a tissue or organ or part thereof. In some embodiments, the cell is a cell that is subject to enhanced apoptosis in an apoptosis mediated disease or condition such as cytopenia, an inflammatory disease, an autoimmune disease, a destructive bone disorder, a proliferative disorder, an infectious disease, a degenerative disease, a disease associated with cell death, an excess dietary alcohol intake disease, a viral mediated disease, uveitis, inflammatory peritonitis, osteoarthritis, pancreatitis, asthma, adult respiratory distress syndrome, glomerulonephritis, rheumatoid arthritis, systemic lupus erythematosus, scleroderma, chronic thyroiditis, Grave's disease, autoimmune gastritis, diabetes, autoimmune hemolytic anemia, autoimmune neutropenia, thrombocytopenia, chronic active hepatitis, myasthenia gravis, inflammatory bowel disease, Crohn's disease, psoriasis, atopic dermatitis, scarring, graft vs host disease, organ transplant rejection, osteoporosis, leukemias and related disorders, myelodysplastic syndrome, multiple myeloma-related bone disorder, acute myelogenous leukemia, chronic myelogenous leukemia, metastatic melanoma, Kaposi's sarcoma, multiple myeloma, haemorrhagic shock, sepsis, septic shock, burns, Shigellosis, Alzheimer's disease, Parkinson's disease, Huntington's disease, prion disease, cerebral ischemia, epilepsy, myocardial, ischemia, acute and chronic heart disease, myocardial infarction, congestive heart failure, atherosclerosis, coronary artery bypass graft, spinal muscular atrophy, amyotrophic lateral sclerosis, multiple sclerosis, HIV-related encephalitis, aging, alopecia, neurological damage due to stroke, ulcerative colitis, traumatic brain injury, spinal cord injury, hepatitis-A, hepatitis-B, hepatitis-C, hepatitis-D, hepatitis-E, hepatitis-G, other forms of viral hepatitis, drug (e.g. paracetamol)-induced liver disease, yellow fever, dengue fever, Japanese encephalitis, liver disease, alcoholic hepatitis, renal disease, polycystic kidney disease, H. pylori-associated gastric and duodenal ulcer disease, HIV infection, tuberculosis, meningitis, and to treat complications associated with coronary artery bypass grafts. The cells affected in these conditions are also tested in the subject methods.

A cell deficient in a protein may be generated by methods known in the art. For example, the technique known as “gene disruption” selectively inactivates a gene in an otherwise normal cell by replacing the gene with a mutant allele. Powerful methods have been developed for accomplishing gene disruption (also called gene knockout) in the cells of organisms such as yeast and mice. These methods rely on the process of homologous recombination, in which regions of sequence similarity exchange segments of DNA. “Homologous recombination” refers to the exchange of nucleic acid regions between two nucleic acid molecules at the site of homologous nucleotide sequences. Foreign DNA inserted into a cell can disrupt any gene with which it is, at least in part, homologous by exchanging segments. Specific genes can be targeted if their nucleotide sequences are known.

In some embodiments the foreign DNA may be located on a targeting construct. A targeting construct is an artificially constructed segment of genetic material which can be transferred into selected cells. The targeting construct can integrate with the genome of the host cell in such a position so as to enhance or inhibit (partially or entirely) expression of a specific gene.

The targeting construct may be produced using standard methods known in the art. For example, as described in Sambrook and Russell, Molecular Cloning: A Laboratory Manual, 3^{rd} Edition, Cold Spring Harbor Laboratory Press, Cold Spring Harbor, N.Y., 2001; Ausubel (ed), Current Protocols in Molecular Biology, 5^{th} Edition, John Wiley & Sons, Inc, NY, 2002.

The development of the targeting construct facilitates its introduction into a cell to be used in a method of the invention. Various techniques for introducing a targeting construct into a host cell, either in vivo or in vitro, are known in the art and include, but are not limited to, microinjection, viral-mediated transfer, and electroporation.

In order to modulate apoptosis and/or the level and/or activity of a member of the Bcl-2 family of proteins, the molecule will be combined with a cell in vitro or in vivo. Combining the molecule and the cell may be achieved by any method known in the art. In some embodiments the cell has been isolated from the organism and combining the molecule and the cell occurs in vitro. In other embodiments the cell has not been isolated from the organism and combining the molecule and the cell occurs in vivo. The molecule may be combined with the cell directly, ie., applied directly to the cell. Alternatively the molecule may be combined with the cell indirectly, eg by injecting the molecule into the bloodstream of an organism, which then carries the molecule to the cell.

A cellular assay may be used to identify molecules which modulate apoptosis and/or the level and/or activity of a member of the Bcl-2 family of proteins. Such methods comprise incubating cells which are sensitive to apoptosis-inducing molecules in the presence of a test molecule and determining the presence of live cells which have not undergone apoptosis. If the molecule modulates the level and/or activity of a member of the Bcl-2 protein family this may be identified by determining whether or not the cell has undergone apoptosis. Alternatively, if the molecule modulates apoptosis of the cell, this may be identified by determining the level and/or activity or a member of the Bcl-2 family of proteins.

Many different methods have been devised to detect apoptosis such as uptake of vital cellular dyes (eosin red, trypan blue, Alamar blue), TUNEL (TdT-mediated dUTP Nick-End Labeling) analysis, ISEL (in situ end labeling), and DNA laddering analysis for the detection of fragmentation of DNA in populations of cells or in individual cells, Annexin-V staining that measures alterations in plasma membranes, detection of apoptosis related proteins such as caspases (by measuring caspase activity or activation), Bcl-2 family proteins, p53, Fas and FADD. These are techniques known to the skilled person.

Similarly, many methods are known to the skilled person for detecting the level and/or activity of a member of the Bcl-2 protein family. For example, the protein can be purified from the cell, such as by chromatographic techniques, and compared to the protein purified from a cell which has not been subjected to the method of the invention.

In some embodiments, the apoptosis-inducing agent is a chemotherapeutic agent. In other embodiments, the agent is a BH3 mimetic agent including: peptides (see for example, Cosulich et al., Current Biology, 7:913-920, 1997; Diaz et al., J. Biol. Chem., 272:11350-11355, 1997; Holinger et al., J. Biol. Chem., 274:13298-13304, 1999; Ottilie et al., Journal of Biological Chemistry, 272:30866-30872, 1997; Schimmer et al., Cell Death Differ., 8:725-733, 2001; Shangary, Biochemistry, 41:9485-9495, 2002; Wang et al., Cancer Research, 60:1498-1502, 2000b); constrained peptides (see for example, Walensky et al., Science, 305:1466-1470, 2004 & WO 2004/058804 incorporated herein in its entirety by reference); foldamers (see for example Sadowsky, J. Am. Chem. Soc., 127(34):11966-11968, 2005); and small organic compounds such as: Antimycin A (e.g. Tzung et al., Nat. Cell. Biol., 3:183-191, 2001); BH3I (e.g. Degterev et al., Nat. Cell. Biol., 3:173-182, 2001); Tetrocarcin A (e.g. Nakashima et al., Cancer Research, 60:1229-1235, 2000); Polyphenols including gossypol, (e.g. Kitada et al., J. Med. Chem., 46:4259-4264, 2003); Apogossypol (e.g. Becattini, 2004); HA14-1 (e.g. Wang et al., Proc. Natl. Acad. Sci. U.S.A., 97:7124-7129, 2000a); Compound 6 (e.g. Enyedy et al., J. Med. Chem., 44:4313-4324, 2001); ABT-737 (e.g. Oltersdorf et al., Nature, 435:677-681, 2005); terphenyl-based compounds (e.g. Yin, J. Am. Chem. Soc., 127(15):5463-5468, 2005); Benzoylurea compounds acting as alpha-helical mimics as disclosed in WO 2006/002474 incorporated herein in its entirety by reference; and Benzothiozole derivatives as disclosed, for example, in U.S. Ser. No. 60/789,982 filed 6 Apr. 2006 incorporated herein in its entirety by reference.

The invention will now be further described by way of reference only to the following non-limiting examples. It should be understood, however, that the examples following are illustrative only, and should not be taken in any way as a restriction on the generality of the invention described above.

### EXAMPLE 1

### Screening for Modulators of Apoptosis and/or the Level and/or Activity of a Member of the Bcl-2 Family of Proteins

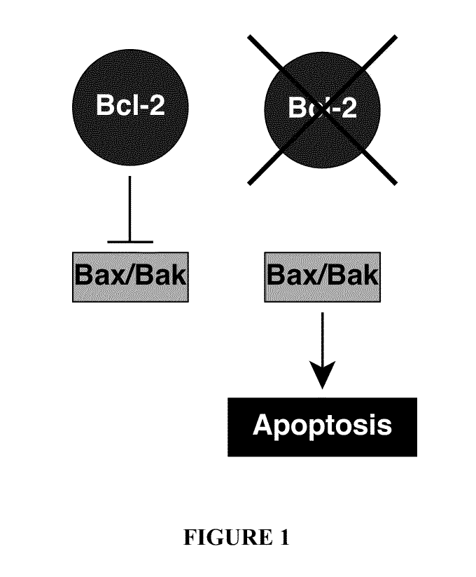

The Bcl-2 family of proteins play a key role in determining whether a cell lives or dies. While there is controversy about some detailed aspects of this system, the essential cell death mediators, Bax and Bak (FIG. 1) are kept in check by the pro-survival proteins (Bcl-2, Bcl-x_{L}, Bcl-w, Mcl-1, A1) until their activity is compromised, usually by antagonistic BH3-only proteins, or in the case of platelets, by the degradation and destruction of Bcl-x_{L}. In this scenario, Bax/Bak are free to act unless restrained by pro-survival Bcl-2-like proteins.

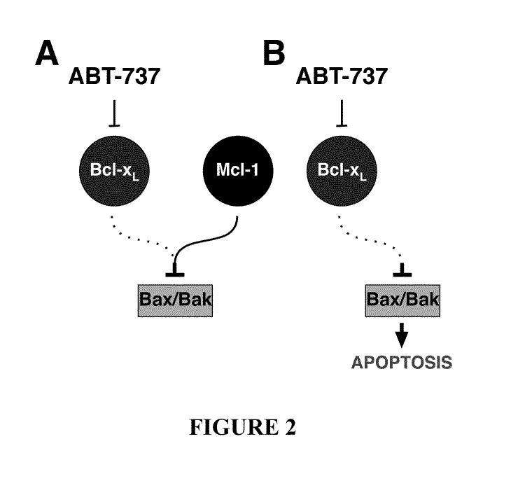

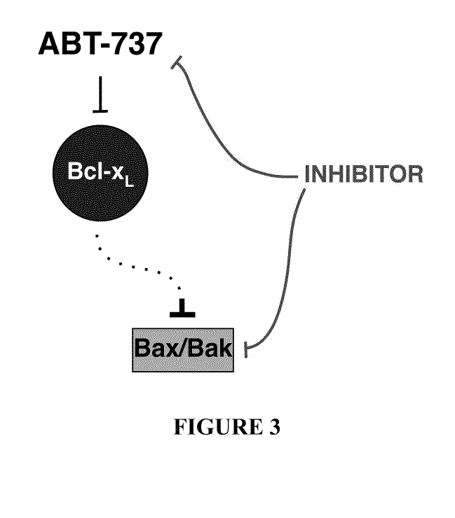

In one embodiment, as shown in FIG. 2, cells are selected or generated in which Bcl-x_{L} is the key control on Bak. Thus, small molecules are screened for those that inhibit cell death even when Bcl-x_{L} is inactivated. This scenario uses mouse embryo fibroblasts lacking Mcl-1, the other control on Bak. In Mcl-1-null cells, the only brake on Bak will be Bcl-x_{L}, which can be abrogated by a compound ABT-737 that acts as a potent inhibitor of Bcl-x_{L}. Compound libraries are screened for those that can block ABT-737-induced killing of Mcl-1 deficient mouse embryo fibroblasts (MEFs) (FIG. 3).

In one non-limiting example, Mcl-1-null MEFs are plated onto flat-bottomed 96-well plates. 12-24 h later, a library compound is added at 0.1, 1 and 10 μM final concentration and incubated for 2 h followed by addition of ABT-737 (100 nM) or a carrier vehicle. Cell viability is scored 24 h later using Alamar Blue dye and read 4 h later. As shown in Table 1, below, the cell viability in the absence of either library compound or ABT-737 acts as a positive control. Lack of cell viability in the presence of no library compound and ABT-737 acts as a negative control. Inert compounds show normal cell viability in the absence of ABT-737. Cytotoxic compounds show reduced cell viability in the presence of library compounds but absence of ABT-737. Positive hits show cell viability in the presence of ABT-737 and a library compound while Negative compounds show reduced cell viability in the presence of a library compound and ABT-737. Positive hits are tested on several independent cell lines and on platelets in culture. In some embodiments, the compounds act by blocking cellular uptake of ABT-737 or inhibiting the action of ABT-737 in cells. In other embodiments, the compounds act by directly inhibiting Bak, Bax, Bak and Bax, or indirectly inhibiting these molecules or apoptosis effector molecules that function downstream of Bak or Bax. The methods are also practised on modified mice with Bcl-2-family genes modified in order to further sensitise the screen and detect the molecular targets of the each positive agent.

### EXAMPLE 2

### Assay of Mcl-1 Null MEF Cells

### 1. Introduction

Apoptosis is induced in Mcl-1^{(-/-)} cells by the compound ABT-737. The cells can be rescued from this effect by the general caspase inhibitor qVD-OPH. The assay aims to discover other compounds that have a comparable effect to that of qVD-OPH.

Rinkenberger et al. (Genes and Development, 14:23-27, 2000) described the generation of Mcl-1-deficient mice. Opferman et al. (Nature, 426:671-676, 2003) described the generation of Mcl-1 conditional knock-out mice and Opferman et al. (Science, 307:1101-1104, 2005) describe their further characterization. Mice generated here are similarly conditionally targeted for the mcl-1 gene. van Delft et al. (Cancer Cell, 10:389-399, 2006) and Lin et al. (Oncogene, 26:3972-3979, 2007) describe how cells deficient for Mcl-1 are very sensitive to ABT-737. Chen et al. (Cancer Research, 67(2):782-791, 2007) show that Mcl-1-null fibroblasts are very sensitive to ABT-737, the basis of the cellular screens.

In summary, cells are split on day one in order to have them at a confluency of 60-80% on day two. On day two, the cells are seeded out into assay plates at a density that will ensure they are not confluent by day four of the assay. The assay plates are incubated at room temperature for 20-60 minutes before being transferred to 37° C. so that edge effects are minimized. For the same reason, assay plates are never stacked on top of each other in the incubator. On day three the cells are treated first with either qVD or with WEHI library compound. The cells are incubated for a 2 hour period in the presence of the library compounds and are then treated with ABT-737. On day four the cells are incubated for four hours in the presence of CellTitre-Blue™ Cell Viability Assay. This product contains resaruzin which is metabolized by live cells to resorufin. After four hours the level of resorufin is measured. Alternatively, in a preferred embodiment, cells are incubated for four hours in the presence of CellTitre-Glo™ Cell Viability Assay. This product measures the level of ATP in the cell culture as a direct correlate of cell viability via a luciferase-dependent luminescence output.

### 2. Reagents, Consumables and Instrumentation

Mcl-1^{(-/-)} mouse embryonic fibroblasts (MEFs) were grown in Iwaki 75 cm² tissue culture flasks (cat #3123-075). MEFs were grown in media consisting of:
89% DME Kelso
10% heat-inactivated foetal calf serum (FCS) (Hyclone cat #SH30396.03)
1% 10 mM asparagine (Fluka cat #11149)
275 μl of a 1:2000 dilution of 2-mercaptoethanol was added to the final 500 ml volume of media (Sigma cat #M7522; diluted in phosphate-buffered saline)

Media was stored at 4° C. and used at 37° C.

MEFs were cultured and harvested using media, phosphate-buffered saline and trypsin (Sigma). All reagents were stored at 4° C. and used at 37° C.

For assays, cells were seeded out in media containing only 1% FCS. This consisted of:
98% DME
1% heat-inactivated foetal calf serum (FCS) (Hyclone cat #SH30396.03)
1% 10 mM asparagine (Fluka cat #11149)
275 μl of a 1:2000 dilution of 2-mercaptoethanol was added to the final 500 ml volume (Sigma cat #M7522; diluted in phosphate-buffered saline)

Assays were seeded out in Corning 384-well tissue culture grade black plates with flat, clear bottoms (DKSH Australia P/L cat #3712). Compounds were made up in Matrical 384-well 50 μl V-bottomed plates (cat #MP101-2-PP). Compound plates were sealed for overnight storage using foil seals from Beckman Coulter (cat #538619).

AnalaR grade DMSO was used for compound preparation and titrations (Merck cat #1.02952.2500). Trypan Blue Solution (0.4%) was used for cell counting (Sigma T8154). CellTitre-Blue™ Cell Viability Assay was sourced from Promega (cat #G8081), stored at −20° C. and used at 37° C. CellTitre-Glo™ which is commercially available from Promega (cat #G7572), is stored at −20° C. and used at 37° C. qVD-OPH general caspase inhibitor was used as a positive control (MP Biomedicals cat. #OPH109).

The Multidrop 384 (ThermolLabsystems) was used to seed the assay plates with cells and to add CellTitre-Blue™ viability reagent to cells. The Zymark Sciclone ALH3000 system was used for control and compound addition. The Wallac EnVision plate reader (Perkin Elmer) was used to measure fluorescence at λₑₓ 535 nm/λₑₘ 590 nm.

### 3. Method

3.1 Day One—Cell Splitting

Media was aspirated off the cells and they were then washed with 10 mls of warm phosphate-buffered saline. The phosphate-buffered saline was aspirated off and 2 ml of trypsin was added to the flask. The flask was placed at 37° C. until the cells were detached. Media (˜6 ml) was used to wash the trypsin and cells to the bottom of the flask. The entire volume was transferred to a 50 ml centrifuge tube and centrifuged for 3 minutes at 250×g. The supernatant was aspirated off and the pellet resuspended in 4 ml of 10% FCS containing media. One millilitre of this cell suspension was added to a clean 75 cm² flask containing 19 ml of 10% FCS containing media, thus performing a 1:4 split. This was repeated with the remaining cell suspension into other 75 cm² flasks, depending on the number of cells required for the following day's assay.

Assay plates for day two were labeled with barcodes.

3.2 Day Two—Seeding Assay Plates and Preparing Control Plates

The protocol for day one was repeated up to the point where the cells were pelleted and the supernatant aspirated off. The pellet was resuspended in 10 mls 1% FCS media. A 1:10 dilution was prepared in a 1.5 ml tube using 800 μl water, 100 μl cell suspension and 100 μl Trypan Blue Solution. The cells were vortexed and then counted using a haemocytometer. The dilution necessary to achieve a density of 2×10⁴ cells ml⁻¹ (1000 cells per well per 50 μl media) in the required volume was calculated and the dilution performed in 1% FCS containing media.

The Multidrop system was used to seed cells into all 384 wells of the assay plates. The system was set up to deliver 50 μl of cell suspension to each well. A sterile cassette head was used and rinsed thoroughly with sterile distilled water before use. The assay plates were rested at room temperature for 60 minutes and then placed at 37° C./5% CO₂ overnight. Plates were not stacked.

The qVD-OPH and ABT-737 plates were set up in Matrical compound plates. qVD was used at a stock concentration of 12.5 mM (final concentration in the cells of 25 μM). 10 μl of this stock was placed in wells I-P in columns 23 and 24. In all remaining wells, 10 μl of DMSO was dispensed. ABT-737 was at a stock concentration of 10 mM and was used at 10 μM in the compound plates (final concentration in the cells of 20 nM). Thus a 1:1000 dilution was performed and then 10 μl was dispensed into all wells of a 384-well Matrical plate except wells 23A-D, 24A-D, 23I-L and 24I-L. DMSO (10 μl) was dispensed into these 16 wells. Alternatively, 10 μl of this stock was placed in wells I-N in columns 20, 21 and 22. In all remaining wells, 10 μl of DMSO was dispensed. WEHI-0113992 was at a stock concentration of 10 mM and was used at 5 μM in the compound plates (final concentration in the cells of 10 nM). Thus a 1:1000 dilution was performed and then 10 μl was dispensed into all wells of a 384-well Matrical plate except wells C20, D₂O, E20, I20, J20, K20, C21, D21, E21, I21, J21, K21, C22, D22, E22, I22, J22, K22. DMSO (10 μl) was dispensed into these 16 wells. Both plates were sealed with foil and stored overnight at 12° C.

3.3 Day Three—Treating the Cells

1. Library plates were removed from the freezer and allowed to thaw at room temperature for 30-60 minutes before use.

2. The Zymark system was set up. The HEPA filter unit was turned on, the pintool was checked to ensure it was clean and unloaded and the deck was set up with blotting paper, ethanol and DMSO as shown below:

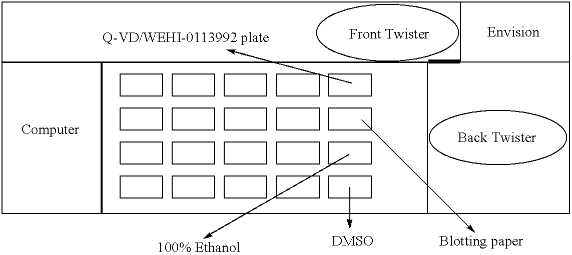

3. The qVD-OPH was added to the cells. To do this, the PVD-OPH plate was placed on the deck (refer to diagram 1) with the A1 corner of the plate facing the corner of the room in which the EnVision computer sits. The assay plates were placed in stack 1 of the front Twister.

Clara Execution Manager was opened on the Zymark desktop PC. All components were initialized by clicking on the “Initialize” button. Once initialization is completed, remove any old applications from the Applications Chain window and add the following ones in the stated order (for each application you need to enter the number of runs i.e. the number of assay plates you are treating):
1. Control Addition
2. Control Addition Restack
3. Pintool Unload

Once these were in place and OKayed the Material Initialization screen came up and was OKayed. One of the Zymark Stack Storage system windows was then brought up and the configuration menu was accessed. “New” was chosen, then “Control Addition” was chosen and OKayed. The configuration menu was again accessed and the process repeated for the “Control Addition Restack” programme.

Once this was completed, the “Run” button on Clara was clicked to begin the run. At the end of the run the assay plates were left in the stacker and the qVD-OPH plate was removed from the Zymark deck.

4. The library compound addition was then begun. The compound plates were placed in stack 1 of the back Twister with A1 facing the MiniTrak. Old applications were removed from the Applications Chain in Clara and the new ones were added in the following order with the number of runs being entered for each application:
1. Pintool Addition Corning
2. Pintool Unload

Once this was done, it was OKayed and the Material Initialization window was checked and OKayed. One of the Zymark Stack Storage system windows was then brought up and the configuration menu was accessed. “New” was chosen, then “Pintool Addition Corning” was chosen and OKayed.

Once this was completed, the “Run” button on Clara was clicked to begin the run.

At the end of the run, the assay plates were re-lidded and returned to 37° C./5% CO₂ for the remainder of the 2 hours (timed from the compound addition to the first assay plate—generally around 30 minutes for a 20 plate run). The library plates were re-lidded and returned to freezer storage.

7. At the end of the 2 hour incubation the ABT-737 addition was carried out. The qVD plate was placed on the deck with the A1 corner of the plate facing the corner of the room in which the EnVision computer sits. The assay plates were placed in stack 1 of the front Twister.

Clara Execution Manager was opened on the Zymark desktop PC. All components were initialized by clicking on the “Initialize” button. Once initialization is completed, remove any old applications from the Applications Chain window and add the following ones in the stated order (for each application the number of runs is entered i.e., the number of assay plates you are treating):
1. Control Addition
2. Control Addition Restack
3. Pintool Unload

Once these were in place and OKayed the Material Initialization screen came up and was OKayed. One of the Zymark Stack Storage system windows was then brought up and the configuration menu was accessed. “New” was chosen, then “Control Addition” was chosen and OKayed. The configuration menu was again accessed and the process repeated for the “Control Addition Restack” programme.

Once this was completed, the “Run” button on Clara was clicked to begin the run. At the end of the run the assay plates were re-lidded and returned to 37° C./5% CO₂ and the ABT-737 plate was removed from the Zymark deck.

8. The HEPA filter was turned off, the DMSO and ethanol reservoirs emptied and washed out and the pintool was cleaned following the protocol below:
Dip 10× in VP cleaning solution; sit for 5 minutes in VP cleaning solution; blot
Dip 10× in MQ water; blot
Dip 10× in 100% ethanol; blot

3.4 Day Four—Viability Analysis

The CellTitre-Glo™ solution was prepared according to the manufacturer's instructions by the reconstitution of CellTitre-Glo™ Substrate with CellTitre-Glo™ Buffer and stored after use at −80° C. Plates were removed from incubator and left to equilibrate to room temperature for 15 mins. 25 μl of diluted CellTitre-Glo™ was added to each well of the assay plates using the Multidrop after removal of 25 μl of cell culture media per well using the MiniTrak. The plates were mixed on a plate shaker for 15 mins before being read on the Envision using the luminescence protocol.

CellTitre-Blue™ was warmed to 37° C. and 10 μl was then added to each well of the assay plates using the Multidrop. The plates were returned to 37° C. for 4 hours before being loaded into the EnVision plate reader. Viability measurements were taken and the data was then imported into ACTIVITYbase (IDBS) for analysis.

For CellTitre-Glo™, the percent inhibition was calculated using the following equation:

$$\% Inhibition = 100 * ( 1 - [ \frac{( x - {μ}^{-} )}{( {μ}^{+} - {μ}^{-} )} ] )$$

x=CPS obtained after sample compound treatment

μ⁻=CPS obtained for the negative controls (columns 24)

μ⁺=CPS obtained for the positive controls (columns 23)

IC₅₀ values were obtained by non-linear least squares fitting of the data using the 4-parameter logistic fit (XLFit 4 eqn #205)

y=A+((B−A)/(1+((C/x)̂D))).

The quality of the assay results were monitored by determination of the Z′ factor for each assay plate, where Z′≧0.5 for the results was considered as robust (Zhang et al., J. Biomol. Screening, 4:67-73, 1999).

### Expected EC50s:

### In 1% FCS:

### ABT-737 EC₅₀˜0.004-0.008 μM

If the calculated EC₅₀ right-shifts to >0.04 μM for ABT-737-treated Mcl(−/−) cells grown under 1% FCS conditions discard MEFs and replenish with newly thawed cells of low passage number

### EXAMPLE 3

### Corticosteroids are Identified in the Subject Screen

A library of known compounds was screened using the protocol set out in Example 2. The results of preliminary analyses indicates several active molecules which were corticosteroids conforming to the following structural formula:

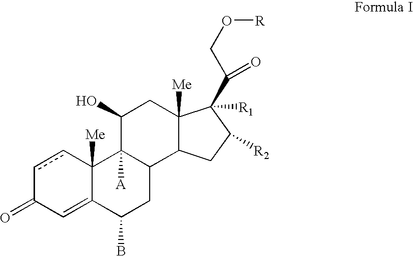

[symbol] can be either a single or double bond

A=H or F, B=H, CH₃, F or OH

R=H or C₂-C₆Acyl

R₁=H, OH or OC₂-C₆Acyl

R₂=H, Me or R₁ and R₂ form a dioxolane ring

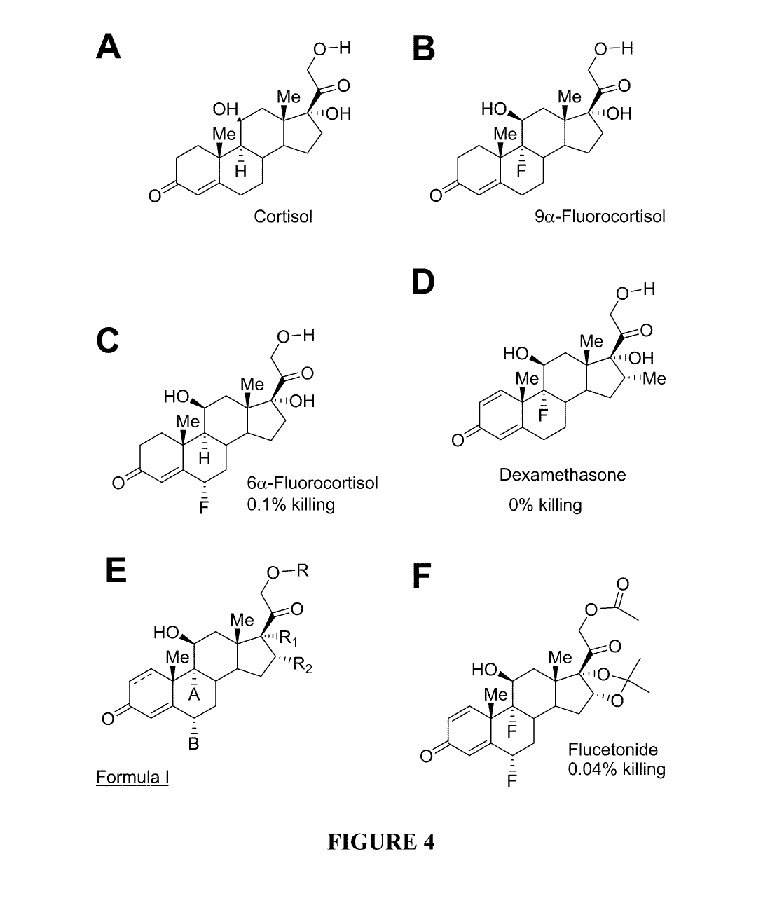

In particular, these agents (see FIG. 4) were able to significantly inhibit killing of Mcl-1 null MEF cells by ABT-737. Accordingly, these agents are suitable for use in the present methods of enhancing or extending platelet viability life span or survival. Further, the agents find broad application in therapeutic interventions to extend or preserve cellular life span.

### EXAMPLE 4

### Protocol for Testing New Anti-Apoptotic Compounds

### Killing Assays (NB: All Experiments Done in the Presence of 10% FCS)

Death stimulus: ABT-737 or Etoposide
For adherent cells (e.g. mcl-1^{-/-} MEFs)
Day 1 Plate out 12,000 cells/24-well in 500 mL media (FMA).
Day 2 Remove media. Add WEHI anti-apoptotic compound in triplicate (4-fold serial dilution—top final concentration of 40 mM in 500 mL). Incubate for 2 hours.
   Add 50 mL of ABT-737 to a final concentration of 40 nM or 50 mL of etoposide to a final concentration of 2 mM to each well.
   Incubate for 24 hours.
Day 3 Harvest cells and stain with propidium iodide, 5 mg/mL in FACS buffer. Determine cell viability by FACs analysis—FLH-3 channel.

For suspension cells (e.g. Jurkats, FDC-P1s)
Day 1 Plate out 20,000 cells/96-well in 100 mL media (HT-RPMI for Jurkats; DME+10% FCS+IL3 for FDC-P1s).
Day 2 Add WEHI anti-apoptotic compound in triplicate (4-fold serial dilution—top final concentration of 40 mM in 100 mL). Incubate for 2 hours.
   Add 20 mL of etoposide to final concentration of 2 mM for FDC-P1s and 10 mM for Jurkats to each well.
   Incubate for 24 hours.
Day 3 Harvest cells and stain with propidium iodide, 5 mg/mL in FACS buffer. Determine cell viability by FACs analysis—FLH-3 channel.

### Death Stimulus: Growth Factor Withdrawal

For growth-factor dependent suspension cells (e.g. FDC-P1s)
Day 1 Plate out 20,000 cells/96-well in 100 mL media (DME+10% FCS). Prior to plating, wash cells first with DME+10% FCS twice to remove IL-3.
Day 2 Add WEHI anti-apoptotic compound in triplicate (4-fold serial dilution—top final concentration of 40 mM in 100 mL). Incubate for 2 hours.
   Incubate for 24 hours.
Day 3 Harvest cells and stain with propidium iodide, 5 mg/mL in FACS buffer. Determine cell viability by FACs analysis—FLH-3 channel.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1

| ABT-737 | Library compound | Cell viability | Comment |
|---|---|---|---|
| − | − | ++ | “Positive control” |
| + | − | − | “Negative control” |
| − | + | ++ | Inert compounds |
| − | + | − | Cytotoxic compounds |
| + | + | ++ | Positive Hit |
| + | + | − | Negatives |

### BIBLIOGRAPHY

Alberts et al., Molecular Biology of the Cell, 4th ed., New York and London: Garland Science, c2002.
Ausubel (ed), Current Protocols in Molecular Biology, 5^{th} Edition, John Wiley & Sons, Inc, NY, 2002.
Chen et al., Cancer Research, 67(2):782-791, 2007.
Cosulich et al., Current Biology, 7:913-920, 1997.
Degterev et al., Nat. Cell. Biol., 3:173-182, 2001.
Diaz et al., J. Biol. Chem., 272:11350-11355, 1997.
Enyedy et al., J. Med. Chem., 44:4313-4324, 2001.
Gait (ed), Oligonucleotide Synthesis, Oxford IRL Press, 1984.
Hames et al. (eds), Nucleic Acid Hybridization, A Practical Approach, IRL Press, 1985.
Holinger et al., J. Biol. Chem., 274:13298-13304, 1999.
Johnson et al., Peptide Turn Mimetics in Biotechnology and Pharmacy, Pezzuto et al., eds Chapman and Hall, New York, 1993.
Kitada et al., J. Med. Chem., 46:4259-4264, 2003.
Lin et al., Oncogene, 26:3972-3979, 2007.
Lodish et al., Molecular Cell Biology”, 4th ed., New York: W. H. Freeman & Co., c2000.
Nakashima et al., Cancer Research, 60:1229-1235, 2000.
Oltersdorf et al., Nature, 435:677-681, 2005.
Opferman et al., Nature, 426:671-676, 2003.
Opferman et al., Science, 307:1101-1104, 2005.
Ottilie et al., Journal of Biological Chemistry, 272:30866-30872, 1997.
Remington's Pharmaceutical Sciences, 17^{th} Edition, Mack Publishing Company, Easton, Pa., USA.
Rinkenberger et al., Genes and Development, 14:23-27, 2000.
Sadowsky, J. Am. Chem. Soc., 127(34):11966-11968, 2005.
Sambrook et al. (eds), Molecular Cloning: A Laboratory Manual”, 2nd Ed., Cold Spring Harbor Laboratory Press; 1989.
Sambrook and Russell, Molecular Cloning: A Laboratory Manual, 3^{rd} Edition, Cold Spring Harbor Laboratory Press, Cold Spring Harbor, N.Y., 2001.
Schimmer et al., Cell Death Differ., 8:725-733, 2001.
Shangary, Biochemistry, 41:9485-9495, 2002.
Tzung et al., Nat. Cell. Biol., 3:183-191, 2001.
van Delft et al., Cancer Cell, 10:389-399, 2006.
Wang et al., Proc. Natl. Acad. Sci. U.S.A., 97:7124-7129, 2000a.
Wang et al., Cancer Research, 60:1498-1502, 2000b.
Yin, J. Am. Chem. Soc., 127(15):5463-5468, 2005.
Zhang et al., J. Biomol. Screening, 4:67-73, 1999.

1. (canceled)

2. (canceled)

3. A method of screening for a molecule which decreases apoptosis of a cell, comprising:
i) combining a candidate molecule with an assay cell;
ii) determining the change in survival of the assay cell in the presence of the molecule relative to a control; and
prior to or between steps i) and ii), treating the assay cell to induce apoptosis with an agent which reduces the level and/or activity of a pro-survival member of the Bcl-2 protein family or an agent which enhances the level and/or activity of a pro-apoptosis member of the Bcl-2 protein family.

4. (canceled)

5. (canceled)

6. The method of claim 3, wherein agent reduces the level and/or activity of Bcl-x_{L} and/or Mcl-1.

7. (canceled)

8. The method of claim 3, wherein agent enhances the level and/or activity of Bak or Bax or Bak and Bax.

9. (canceled)

10. (canceled)

11. (canceled)

12. (canceled)

13. (canceled)

14. (canceled)

15. (canceled)

16. The method of claim 3, wherein the assay cell is modified to enhance its sensitivity to an apoptosis inducing agent.

17. The method of claim 16, wherein the assay cell is genetically modified to reduce the level or activity of at least one pro-survival member of the Bcl-2 family.

18. The method of claim 17, wherein the cell is an Mcl-1 deficient cell.

19. The method of claim 18, wherein the agent reduces the level or activity of Bcl-x_{L}.

20. The method of claim 18, wherein the agent is a BH3 domain mimicking agent.

21. (canceled)

22. (canceled)

23. (canceled)

24. The method of claim 3, wherein the candidate molecule is a small molecule, inhibitory RNA, antibody, aptamer, peptide, peptidomimetic or constrained peptide.

25. (canceled)

26. The method of claim 16, further comprising:
iii) combining the candidate molecule with a cell and determining the change in survival of the cell in the presence of the molecule relative to a control.

27. The method of claim 26, wherein the cell is a mammalian cell.

28. The method of claim 27, wherein the mammalian cell is a myeloid cell, lymphoid cell, neural cell, epithelial cell, endothelial cell, stem or progenitor cell, hepatocyte, myoblast, osteoblast, osteoclast, lymphocyte, keratinocyte, melanocyte, mesothelial cell, germ cell, muscle cell, fibroblast, a transformed cell, a cancer cell.

29. The method of claim 27, wherein the mammalian cell is a cell subject to enhanced apoptosis in an apoptosis mediated disease or condition.

30. The method of claim 3, further comprising:
iii) combining the candidate molecule with a cell and determining the change in survival of the cell in the presence of the molecule relative to a control.
